(19) United States
(12) Patent Application Publication
(10) Pub. No.: US 2013/0157368 A1
(43) Pub. Date: Jun. 20, 2013

(54) INDUCED PLURIPOTENT STEM CELLS PREPARED FROM HUMAN KIDNEY-DERIVED CELLS

(71) Applicants: Charito Buensuceso, North Brunswick, NJ (US); Agnieszka Seyda, Belle Mead, NJ (US); David C. Colter, Hamilton, NJ (US); Sridevi Dhanaraj, Raritan, NJ (US); Brian C. Kramer, Plainfield, NJ (US); Jason Elliot Ekert, Jeffersonville, PA (US); Amanda Lynn Kauffman, Morrisville, PA (US)

(72) Inventors: Charito Buensuceso, North Brunswick, NJ (US); Agnieszka Seyda, Belle Mead, NJ (US); David C. Colter, Hamilton, NJ (US); Sridevi Dhanaraj, Raritan, NJ (US); Brian C. Kramer, Plainfield, NJ (US); Jason Elliot Ekert, Jeffersonville, PA (US); Amanda Lynn Kauffman, Morrisville, PA (US)

(73) Assignee: Advanced Technologies and Regenerative Medicine, LLC, Raynham, MA (US)

(21) Appl. No.: 13/331,283

(22) Filed: Dec. 20, 2011

(51) Int. Cl. C12N 5/071; C12N 15/867

(52) U.S. Cl. 435/456; 435/369

(57) ABSTRACT

We have disclosed an induced pluripotent stem cell and the method of preparing the induced pluripotent stem cell from a human kidney-derived cell. More particularly, we have disclosed a human kidney-derived iPS cell which may be differentiated into cells of ectoderm, mesoderm, and endoderm lineages.

### FIELD OF THE INVENTION

The invention relates to induced pluripotent stem cells. More particularly, the invention relates the reprogramming of human kidney-derived cells (hKDC) into induced pluripotent stem (iPS) cells.

### BACKGROUND OF THE INVENTION

Induced pluripotent stem (iPS) cells have generated interest for application in regenerative medicine, as they allow the generation of patient-specific progenitors in vitro having a potential value for cell therapy (Takahashi, K. and Yamanaka, S., Cell 126, 663-76 (2006)). However, in many instances an off-the-shelf approach would be desirable, such as for cell therapy of acute conditions or when the patient's somatic cells are altered as a consequence of a chronic disease or ageing. Ectopic expression of pluripotency factors and oncogenes using integrative viral methods is sufficient to induce pluripotency in both mouse and human fibroblasts (Takahashi, K. and Yamanaka, S., Cell 126, 663-76 (2006); Takahashi, K. et al. Cell 131, 861-72 (2007); Hochedlinger, K. and Plath, K., Development 136, 509-23 (2009); Lowry, W. E. et al., Proc Natl Acad Sci USA 105, 2883-8 (2008)). However, this process is slow, inefficient and the permanent integration of the vectors into the genome limits the use of iPS cells for therapeutic applications (Takahashi, K. and Yamanaka, S., Cell 126, 663-76 (2006)). Further studies have shown that the age, origin, and cell type used has a deep impact on the reprogramming efficiency. Recently, it was shown that retroviral transduction of human keratinocytes resulted in reprogramming to pluripotency which was 100-fold more efficient and twice as fast when compared to fibroblasts. It was hypothesized that these differences could result from the endogenous expression of KLF4 and c-MYC in the starting keratinocyte population and/or the presence of a pool of undifferentiated progenitor cells presenting an epigenetic status more amenable to reprogramming (Lowry, W. E. et al., Proc Natl Acad Sci USA 105, 2883-8 (2008).). This latter hypothesis has been further supported by other studies in mouse. (Silva, J. et al., PLoS Biol 6, e253 (2008); and Eminli, S. et al., Stem Cells 26, 2467-74 (2008)). However, stem cells are usually rare and difficult to access and isolate in large amounts (e.g., neural stem cells) (Kim, J. B. et al., Cell 136, 411-9 (2009); Kim, J. B. et al., Nature 454, 646-50 (2008)).

Human kidney-derived iPS cells represent a viable supply of pluripotent cells for a number of applications. For example, the iPS cells obtained from patients suffering from genetic kidney or other renal disorders can be used for disease modeling in order to understand the development of the disease. Human kidney-derived iPS cells can be differentiated into renal cells and hepatocytes for cell replacement and transplantation therapies in renal and liver diseases, respectively. In addition, renal cells and hepatocytes differentiated from human kidney-derived iPS cells are ideal for screening compounds for evaluating their efficacy and toxicology with regards to specific kidney and liver disease conditions.

### SUMMARY OF THE INVENTION

We describe herein, an induced pluripotent stem cell prepared by reprogramming a human kidney-derived cell wherein the human kidney-derived cell is positive for the expression of HLA-I and CD 44 and at least one of Oct-4, Rex-1, Pax-2, Cadherin-11, FoxD1, WT1, Eya1, HNF3B, CXC-R4, Sox-17, EpoR, BMP2, BMP7, or GDF5; and negative for the expression of CD133 and E-cadherin and at least one of Sox2, FGF4, hTert, Wnt-4, SIX2 or GATA-4.

### BRIEF DESCRIPTION OF THE FIGURES

FIG. 1. Morphology of human kidney-derived iPS cells obtained from transduction of hKDC with human OCT4, SOX2, KLF4, and c-MYC. Clones are shown on irradiated mouse embryonic fibroblast (MEF) feeder layer at passage 1.

FIG. 2. Morphology of human kidney-derived iPS cells obtained from transduction of hKDC with human OCT4, SOX2, KLF4, and c-MYC and shRNA to p53. Clones are shown on irradiated mouse embryonic fibroblast (MEF) feeder layer at passage 1.

FIG. 3. Human kidney-derived iPS cells (clone RV4-5) grown on MATRIGEL and stained for alkaline phosphatase (4× magnification).

### DETAILED DESCRIPTION OF THE INVENTION

We disclose herein, the reprogramming of human kidney-derived cells (hKDC) to pluripotency by retroviral transduction of four (OSKM) transcription factors with or without the downregulation of p53. Using the methods and compositions described herein, hKDC are reprogrammed to pluripotency by retroviral transduction with OCT4, SOX2, KLF4, and c-MYC. The resulting reprogrammed hKDC have the characteristics of an induced pluripotent stem (iPS) cell.

In one embodiment, an induced pluripotent stem (iPS) cell is prepared from a human kidney-derived cell, referred to herein as a human kidney-derived iPS cell. The hKDC were reprogrammed by the forced expression of the reprogramming factors in the presence or absence of shRNA to p53. The reprogrammed cells were characterized for morphology, staining for alkaline phosphatase, expression of pluripotency markers, methylation of specific promoters, and expression of specific germ layer markers.

hKDC are a unique population of cells isolated from human cadaveric kidney tissue. The methods for isolating hKDC are described in pending US Patent Publication Number 2008/0112939, incorporated by reference herein in its entirety. Briefly, these cells were isolated by obtaining tissue from the subcapsular, cortex, or medulla region of a mammalian kidney. Fragmented kidney tissue was incubated in the presence of a metalloprotease, a neutral protease, or a mucolytic enzyme and the cells were plated in a tissue culture vessel.

The isolated or purified human kidney-derived cell population is capable of self-renewal and expansion in culture. The cell population is positive for expression of HLA-I and CD 44 and at least one of Oct-4, Rex-1, Pax-2, Cadherin-11, FoxD1, WT1, Eya1, HNF3B, CXC-R4, Sox-17, EpoR, BMP2, BMP7, or GDF5; and negative for the expression of CD133 and E-cadherin and at least one of Sox2, FGF4, hTert, Wnt-4, SIX2 or GATA-4.

In addition, the cells are positive for expression is positive for at least one of cell-surface markers CD24, CD29, CD49c, CD73, CD90, CD166, or SSEA-4; and negative for at least one of cell-surface markers HLA 11, CD31, CD34, CD45, CD56, CD80, CD86, CD104, CD105, CD117, CD138, and CD141.

The human kidney-derived cell population secretes at least one of trophic factors FGF2, HGF, TGFα, TIMP-1, TIMP-2, MMP-2 or VEGF; and does not secrete at least one of trophic factors PDGF-bb or IL12p70.

The hKDCs may be reprogrammed using conventional reprogramming techniques including, viral, such as adenoviral, lentiviral, and retroviral; chemical, such as small molecule mimicking; proteins, such as recombinant proteins; RNA, such as microRNA and messenger RNA (mRNA); and vectors.

In one embodiment, the hKDC were reprogrammed using viral reprogramming methods. In one embodiment, the hKDC were transfected with VSVg murine retroviruses individually carrying constitutively expressed human transcription factors OCT4, SOX2, KLF4, and c-MYC. Briefly, hKDC were plated in a 6-well plate, at 1×10⁵ cells per well in renal epithelial growth medium (REGM), and incubated overnight at 5% CO₂ and 37° C. For viral transfections, transduction medium having the four VSVg murine retroviral constructs (OCT4, SOX2, KLF4, and c-MYC) and an agent for increasing the efficiency of transfection was prepared for each well. Medium was aspirated from the wells, transduction medium was added, and incubated overnight at 5% CO₂ and 37° C. This transduction step was repeated the following day and after overnight incubation, the transduction medium was replaced with REGM. Cells were allowed to incubate for another four days with REGM replaced every two days.

Optionally, the transduction medium also included the VSVg murine retrovirus carrying p53-shRNA. The inhibition of p53 has been previously shown to enhance the reprogramming efficiency of specific cell types presumably by slowing down cell proliferation (Zhao Y et al., (2008) Cell Stem Cell 3: 475-479; Sarig, R., et al., J. Exp. Med. 207: 2127-2140 (2010)). The transfected hKDC were then cultured and observed for the appearance of classical iPS cell morphology. Classical iPS cell morphology refers to the formation of tightly packed cell colonies that are refractive or “shiny” under light microscopy with very sharp and well-defined edges. Cells exhibiting classical IPS cell morphology were isolated, subcultured, and expanded to provide human kidney-derived iPS cells.

In another embodiment, the hKDC were reprogrammed using mRNA encoding for the transcription factors OCT4, KLF4, SOX2, C-MYC, and LIN28. Briefly, hKDC were plated in a 6-well plate in REGM and incubated overnight at 5% CO₂ and 37° C. For mRNA transfections, mRNA transfection complex containing the five human mRNA (OCT4, SOX2, KLF4, c-MYC, and LIN28) and an agent for increasing the efficiency of transfection was prepared. The REGM medium was aspirated from the wells, transduction medium was added, after fours, the transduction medium was replaced with a reprogramming medium and incubated overnight at 5% CO₂ and 37° C. This transduction step was repeated daily for sixteen days. Cells were monitored for iPS cell colonies with daily medium changes.

Several criteria are used to assess whether iPS cells are fully reprogrammed including morphology (as described above), staining for alkaline phosphatase, expression of pluripotency markers, methylation of specific promoters, and expression of specific germ layer markers. The expression of key pluripotency factors (OCT4, NANOG) and embryonic stem cell specific surface antigens (SSEA-3, SSEA-4, TRA1-60, TRA1-81) have been routinely used to identify fully reprogrammed human cells. At the functional level, iPS cells also demonstrate the ability to differentiate into lineages from all three embryonic germ layers.

The human kidney-derived iPS cell prepared by the methods described herein was characterized for pluripotency. These cells which display the classical iPS cell morphology, are capable of self-renewal, express the key pluripotency markers (TRA1-60, TRA1-81, SSEA3, SSEA4, and NANOG), demonstrate differentiation into lineage from three germ layers, and show normal karyotype.

Human kidney-derived iPS cells represent a good source of pluripotent cells for regenerative medicine. With this technology, it is now possible to generate pluripotent cells in large numbers. Another important benefit is the potential to obtain disease-specific human kidney-derived iPS cells from patients with genetic kidney disease such as polycystic kidney disease (PKD) and Alport Syndrome. Reprogrammed cells derived from patients with PKD and Alport Syndrome that maintain the disease genotype and phenotype indefinitely could be used for disease modeling and screening compounds aimed at modifying epigenetic and/or transcriptional abnormalities, important regulators of these genetic disorders. In addition, such PKD and Alport patient-derived iPS lines could be generated to correct the genetic defect identified in the cells.

Reprogrammed hKDC that have been differentiated into hepatocyte-like cells have great therapeutic potential for regenerative medicine and for liver disease. Acute liver failure (ALF) is a devastating clinical syndrome occurring approximately 2000 cases per year in the US and is associated with a mortality reaching 80%. Currently, orthotopic liver transplantation is the only available therapy showing survival rates from 70% to 85%. A cell-based therapy could be a potential solution as cellular transplantation using primary hepatocytes has been used successfully in rodent and human models. Hepatocytes derived from human kidney-derived iPS cells represent a potential source of transplantable cells for promoting normal liver function in diseased livers.

The invention is further explained in the description that follows with reference to the drawings illustrating, by way of non-limiting examples, various embodiments of the invention.

### EXAMPLES

### Example 1

### Viral Reprogramming of hKDC into iPS Cells

hKDC obtained according to the methods described in US Patent Publication Number 2008/0112939 were transduced with retroviral constructs from Stemgent, Inc. (San Diego, Calif.), specifically VSVg murine retroviruses individually carrying constitutively expressed human transcription factors (OCT4, SOX2, KLF4, and c-MYC) with or without VSVg murine retrovirus containing p53-shRNA.

The murine retroviruses were produced using the 293-gp2 retrovirus packaging cells that were plated one day prior to transfection onto 6 centimeter dishes at a density of 3×10⁶ cells per dish and incubated overnight at 5% CO₂ and 37° C. Each dish was then transfected with 3 micrograms of a single pMX vector (Sox2, Oct4, cMyc or Klf4, or p53-shRNA), 1 microgram VSV-g and 16 microliters of transfection agent sold under the tradename FUGENE HD (Roche Applied Bioscience, Indianapolis, Ind., catalog number 04709705001) according to the manufacturer's standard protocol. Viruses were then collected 48 hours after transfection and filtered through a 0.45 micron filter prior to use.

hKDC were thawed and cultured for one passage before transduction. One day before transduction, hKDC were trypsinized and plated onto 2 wells of a 6-well plate at 1×10⁵ cells per well in 2 milliliters of renal epithelial growth medium (REGM, Lonza LTD. Corporation, Walkersville, Inc., Walkersville, Md.) per well. Cells were incubated overnight at 5% CO₂ and 37° C. On day 1, 2.5 milliliters of transduction medium was prepared for each well containing 500 microliters of each freshly-made virus (OCT4, KLF4, SOX2, and C-MYC) and 4 nanograms/milliliter of polybrene. The culture medium was aspirated from the wells, the transduction medium was added, and was incubated overnight at 5% CO₂ and 37° C. On day 2, the viral transduction step was repeated. On day 3, the transduction medium was removed and replaced with REGM. Media changes were performed every 2 days until day 7.

To monitor the formation of reprogrammed or iPS cell colonies, the transduced hKDC were harvested by trypsinization, resuspended in culture medium sold under the tradename STEMEDIUM NUTRISTEM (Stemgent, Inc., Cambridge, Mass., catalog number 01-0005) supplemented with an additional 20 nanograms/milliliter of basic fibroblast growth factor (bFGF) (iPS-Nu medium) or standard knockout serum replacement (KSR)-containing human ES medium with 20 nanograms/milliliter of bFGF (iPS-KSR medium), and then plated on a basement membrane matrix, sold under the tradename MATRIGEL (BD Biosciences, Chicago, Ill., catalog number 354277)-coated or mouse embryonic fibroblast (MEF) feeder plate at a concentration of 1×10⁴ cells per well in 6-well plate. Medium was changed with fresh iPS cell medium every 2 days during the first week and daily during weeks 2 to 6. The plates were checked daily to identify iPS cell colonies.

Colonies exhibiting the ‘classic’ reprogrammed or iPS cell morphology were manually picked from MEF feeder plates and seeded onto a single well of a 12-well MEF feeder plate. Culture medium was changed daily. After 4-6 days, the colonies were manually picked from the 12-well plates and expanded into 6-well plates. Culture medium was changed daily and manually split 1:3 every 4-6 days. Cells from each well were frozen at various stages using a freezing medium, sold under the tradename CRYOSTEM (Stemgent, Inc., catalog number 01-0013).

### Results

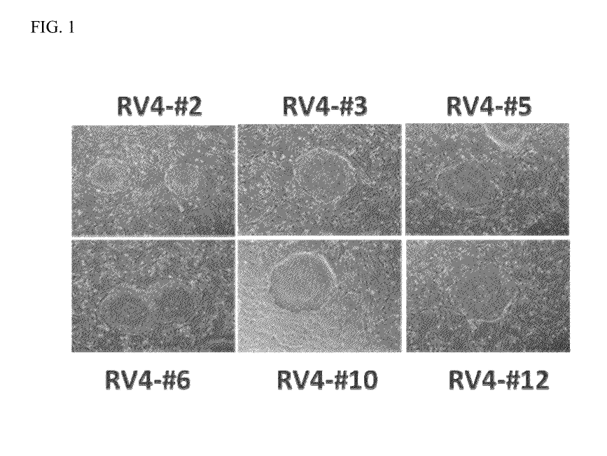

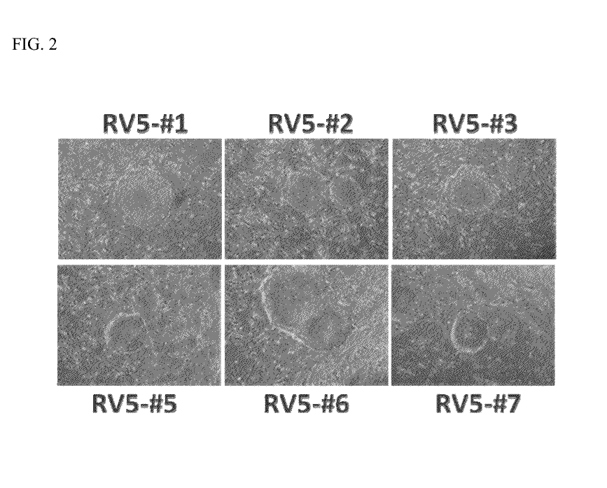

Reprogramming of hKDC with the retroviruses expressing the four reprogramming factors resulted in colonies exhibiting the iPS cell morphology. Twelve reprogrammed colonies obtained from the viral transduction with the four reprogramming factors, denoted as RV4 followed by the colony number, were manually picked and of these colonies, 6 were expanded and frozen (FIG. 1). For the reprogramming of hKDC with the reprogramming factors and shRNA to p53, denoted as RV5 followed by the colony number, 12 colonies were manually picked and 6 were expanded and frozen (FIG. 2).

### Example 2

### Expression of Pluripotency Markers

The human kidney-derived iPS cells prepared in Example 1 were assessed for the expression of pluripotency markers by immunocytochemistry. Following fixation of the colonies in 4% paraformaldehyde, immunofluorescent staining for pluripotency markers was performed using the antibody reagents shown in Table 1 (all antibodies were obtained from Stemgent, Inc.).

TABLE 1

| Marker | Primary Antibody | Secondary Antibody |
|---|---|---|
| TRA-1-81 | Mouse anti-Human TRA-1-81 | NA |
| | Antibody, sold under the | |
| | tradename DYLIGHT 549, | |
| | catalog number 09-0082 | |
| TRA-1-60 | Mouse anti-Human TRA-1-60 | NA |
| | Antibody, sold under the | |
| | tradename STAINALIVE | |
| | DYLIGHT 488, catalog | |
| | number 09-0068 | |
| SSEA-3 | Anti-Human SSEA-3 | Goat anti-Rat IgG + IgM |
| | Antibody, catalog number | Antibody, sold under the |
| | 09-0014 | tradename CY 3, catalog |
| | | number 09-0038 |
| SSEA-4 | Anti-Human SSEA-4 | Goat anti-Mouse IgG + IgM |
| | Antibody, catalog number | Antibody, sold under the |
| | 09-0006 | tradename CY 3, catalog |
| | | number 09-0036 |
| NANOG | Anti-Mouse/Human NANOG | Goat anti-Rabbit IgG |
| | Antibody, catalog number | Antibody, sold under the |
| | 09-0020 | tradename CY 3, catalog |
| | | number 09-0037 |

### Results

Two representative human kidney-derived iPS cell clones were assessed for expression of pluripotency markers. The human kidney-derived iPS cell clones tested, RV4-5 and RV5-1, both express the markers TRA1-60, TRA1-81, SSEA3, SSEA4, and NANOG. These markers were not detected in the parental hKDC. The expression of these markers indicates pluripotency of the human kidney-derived iPS cells.

### Example 3

### Methylation Analysis of Oct4, Nanog, and Sox2 Promoters

The human kidney-derived iPS cells prepared in Example 1, clones RV4-5 and RV5-1, were analyzed for the methylation status of the Oct4, Nanog, and Sox2 promoter regions using the bisulfite sequencing method and analysis was performed by Seqwright, Inc. (Houston, Tex.). The bisulfite method is the most commonly used technique for identifying specific methylation patterns within a DNA sample. It consists of treating DNA with bisulfite, which converts unmethylated cytosines to uracil but does not change methylated cytosines. It is used both for loci-specific or genome-wide analyses.

Approximately 100 to 500 bp-long promoter regions of Oct4, Nanog, and Sox2 were examined for methylation patterns. DNA (see Table 2) were prepared using the DNA extraction kit sold under the tradename DNEASY (Qiagen, Inc., Valencia, Calif., catalog number 69506) and were sent to Seqwright, Inc. for analysis.

TABLE 2

| Sample ID | Sample description |
|---|---|
| 1 | parental hKDC |
| 2 | hKDC RV5-1 p17 |
| 3 | hKDC RV4-5 p14 |

### Results:

Table 3 summarizes the results obtained from the methylation analysis of the promoter regions. Within the regions tested, no methylation sites were detected within the Nanog and Sox2 promoter regions. For the Oct4 promoter region, 7 methylation sites were detected. Both clones of human kidney-derived iPS cells showed a change in the methylation of these 7 sites relative to the parental cells. Changes in methylation pattern relative to the parental cells is characteristic of iPS cells.

TABLE 3

| | | Total | | |
|---|---|---|---|---|
| | | methylation sites | Total | Total |
| | Bp | found in the | changed | unchanged |
| Promoter region | examined | region | sites | sites |
| Oct4 promoter | ~520 bp | 7 | 7 | 0 |
| Nanog promoter | ~100 bp | 0 | 0 | 0 |
| Sox2 promoter | ~550 bp | 0 | — | — |

### Example 4

### Alkaline Phosphatase Staining

The pluripotency of the human kidney-derived iPS cells prepared in Example 1, clone RV4-5, was also assessed by alkaline phosphatase (AP) staining and was performed using an alkaline phosphatase detection kit (Millipore Corporation, Billerica, Mass., catalog number SCR004). Human kidney-derived iPS cells were plated onto 24-well plates and maintained in a 37° C. incubator. After 3-5 days, culture media was aspirated from the wells and the cells were fixed using 4% paraformaldehyde for 1-2 minutes. The fixative was removed and the cells were washed with 1 milliliter of 1× rinse buffer. Afterwards, rinse buffer was replaced with 0.5 milliliter of staining reagent mix and incubated at room temperature for 15 minutes. The staining reagent was prepared by mixing the kit components fast red violet (FRV) and naphthol AS-BI phosphate solution with water in a 2:1:1 ratio (FRV:Naphthol:water) in an aluminum foil-covered tube. The staining reagent was removed and cells were washed once with 1 milliliter of 1× rinse buffer and then incubated in 0.5 milliliter of PBS. Images of stained cells were captured with a photomicroscope. Cells exhibiting AP activity appear purple.

### Results

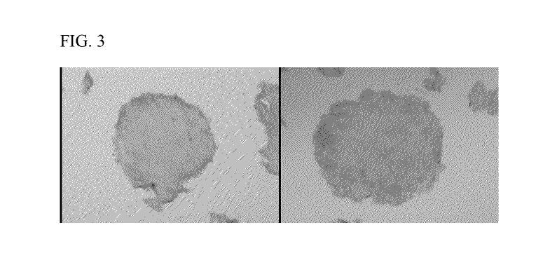

As shown in FIG. 3, human kidney-derived iPS cells, clone RV4-5, exhibited positive alkaline phosphatase staining that is indicative of the pluripotent state.

### Example 5

### Differentiation into Lineages of Three Germ Layers

The differentiation capacity of the human kidney-derived iPS cells prepared in Example 1, clone RV5-1, into ectodermal, mesodermal, and endodermal lineages was evaluated by inducing embryoid body formation and staining for markers specific to the three germ layers.

Embryoid bodies were generated using clustering plates, sold under the tradename AGGREWELL 400 (STEMCELL Technologies, Inc., Vancouver, Canada, catalog number 27940). Cells were enzymatically dissociated using a cell detachment solution, sold under the tradename ACCUTASE (STEMCELL Technologies, Inc.), resuspended in MEF conditioned medium (GlobalStem, Incorporated, Rockville, Md. catalog number GSM-9100) supplemented with 100 nanograms/milliliter bFGF, and counted by trypan blue staining using a hemocytometer. To induce embryoid body formation, 0.5 to 1 million cells were added to each well of an AGGREWELL 400 plate and the plate was centrifuged at 1000 rpm for 5 minutes to capture the cells in the microwells. After incubation at 37° C. in 5% CO₂ and 95% humidity for 24 hours, the embryoid bodies were harvested by aspiration and passing the suspension through an inverted 40 micron cell strainer on top of a 50 milliliter conical tube to remove single cells. The aggregates remained on top of the inverted cell strainer and were collected by washing the aggregates off from the cell strainer using MEF conditioned medium. Embryoid bodies were then plated onto low cluster plates. The medium was changed into a 1:1 mixture of MEF conditioned medium and DMEM/F12 after 24 hours and kept in culture for 7 days prior to staining for markers of germ layer differentiation.

Immunocytochemistry of the differentiated human kidney-derived iPS cells was performed by fixing the cells in 4% paraformaldehyde in phosphate-buffered saline (PBS) pH 7.4 for 15-20 minutes at room temperature and washing with ice-cold PBS. The cells were incubated with 10% normal donkey or goat serum in PBS at room temperature for 1 hour to block non-specific binding of the antibodies. Afterwards, the cells were incubated in the specific antibody (Table 4) in 10% goat serum in PBS in a humidified chamber for 2 hours at room temperature or overnight at 4° C. Cells were washed with PBS and then incubated with the secondary antibody for 1.5-2 hours at room temperature in the dark. After washing the cells with PBS, cell nuclei were visualized by incubating the cells in 0.1-1 microgram/milliliter API (DNA stain, 1:10000 diluted) for 2 minutes. After a final wash with PBS, the cells were processed for immunofluorescence microscopy.

TABLE 4

| Germ | | |
|---|---|---|
| Layer | Primary Antibody | Secondary Antibody |
| Ectoderm | Nestin (Stemgent Inc, | Goat anti-mouse IgG antibody, |
| | catalog number 09-0045) | sold under the tradename |
| | | ALEXA FLUOR 680, |
| | | (Invitrogen Corporation, |
| | | Carlsbad, CA, catalog number |
| | | A20983) |
| Mesoderm | Alpha-smooth muscle actin | Goat anti-mouse IgG antibody, |
| | (SMA; Sigma-Aldrich, St. | sold under the tradename |
| | Louis, MO, catalog number | ALEXA FLUOR 680, |
| | SAB1400414) | (Invitrogen Corporation, |
| | | catalog number A20983) |
| Endoderm | Alpha-fetoprotein1 (AFP1; | FITC Goat anti-rabbit IgG |
| | Dako North America, Inc., | (Abcam, Plc., Cambridge, MA, |
| | Carpinteria, CA, catalog | catalog number Ab6717) |
| | number A0008) | |

### Results

The human kidney-derived iPS cells were stained with antibodies to nestin, alpha-smooth muscle actin (alpha-SMA), and alpha-fetoprotein 1 (AFP1) to evaluate differentiation into ectodermal, mesodermal, and endodermal lineages, respectively. The human kidney-derived iPS cell, clone RV5-1, expressed these germ layer markers after embryoid body formation indicating that these cells have the capacity to differentiate into cells from these germ layers.

### Example 6

### Differentiation into Hepatic Lineage

The differentiation of human kidney-derived iPS cells prepared in Example 1 into cells of the hepatic lineage was performed using modifications of published protocols (Hay, D. et al., Proc Natl Acad Sci USA. 105(34):12301-6 (2008)).

Human kidney-derived iPS cells, clone R4-5, weaned off from feeder layer were cultured on MATRIGEL and maintained in mouse embryonic fibroblast (MEF) conditioned medium (GlobalStem, Incorporated, catalog number GSM-9100) containing 100 nanogram/milliliter bFGF (Millipore Corporation, Billerica, Mass., catalog number GF003). 10 micromolar Rho kinase (ROCK) inhibitor (EMD Chemicals, Inc., Gibbstown, N.J., Catalog number 668000) is included in culture medium only on the first day after passaging.

When the human kidney-derived iPS cells reached about 50-70% confluency, MEF conditioned medium was replaced with RPMI1640 medium (Invitrogen Corporation, catalog number 21870092) containing 1× concentration of a serum-free supplement sold under the tradename B27 SUPPLEMENT (Invitrogen Corporation, catalog number 17504044), 2 mM of the L-glutamine alternative sold under the tradename GLUTAMAX (Invitrogen Corporation, catalog number 35050-061), 100 nanograms/milliliter activin A (R&D Systems, Inc., Minneapolis, Minn., catalog number 338-AC-050) and 50 nanograms/milliliter Wnt3a (R&D Systems, Inc., catalog number 5036-WN-010) for 72 hours.

The cells were then split 1:2 to new MATRIGEL-coated plates and cultured in differentiation medium: knockout-Dulbecco's modified Eagle's medium (DMEM; Invitrogen Corporation, catalog number 10829-018) containing 20% serum replacement (SR; Invitrogen Corporation, catalog number 10828010), 1 millimolar GLUTAMAX L-glutamine alternative, 1% non-essential amino acids (Invitrogen Corporation, catalog number 11140050), 0.1 millimolar beta-mercaptoethanol (Sigma-Aldrich, catalog number M7522) and 1% dimethyl sulfoxide (DMSO) (Sigma-Aldrich, catalog number S2650), for 7 days. Finally, the cells were cultured in maturation medium: Leibovitz's L15 medium (Invitrogen Corporation, Catalog number 11415) supplemented with 8.3% fetal bovine serum sold under the tradename HYCLONE FBS (Thermo Fisher Scientific, Inc., Waltham, Mass., catalog number SH30070.031), 8.3% tryptose phosphate broth (Sigma-Aldrich, catalog number T8159), 10 micromolar hydrocortisone 21-hemisuccinate (Sigma-Aldrich, catalog number H2882), 1 micromolar insulin (Sigma-Aldrich, catalog number 19278), 2 millimolar GLUTAMAX L-glutamine alternative, 10 nanograms/milliliter hepatocyte growth factor (HGF; R&D Systems, Inc., catalog number 294-HG-005) and 20 nanograms/milliliter oncostatin M (OSM; R&D Systems, Inc., catalog number 295-OM-010) for 7 days. The medium was changed daily during differentiation.

The expression of hepatic markers was assessed by qPCR. RNA was prepared using a RNA and protein extraction kit, sold under the tradename ALLPREP RNA/Protein Kit (Qiagen, Inc., Catalog number 80404), according to manufacturer's instruction. The amount of lysis buffer used for cells grown in 6 well-plates was scaled up accordingly.

To prepare samples for qPCR, genomic DNA removal was performed according to manufacturer's instruction. cDNA synthesis was performed with 0.5 micrograms of total RNA isolated from human kidney-derived iPS cells or differentiated cells using the cDNA synthesis kit sold under the tradename QUANTITECT Reverse Transcription Kit (Qiagen, Inc., catalog number 205313) in a total volume of 20 microliters. PCR was performed in a 7300 Real time PCR System in optical 96-well reaction plates sold under the tradename MICROAMP (Applied Biosystems, Inc., Carlsbad, Calif., catalog number 4306737) in a final volume of 20 microliters. Human transcripts, were detected with 10 microliters of 2×PCR reaction mix sold under the tradename TAQMAN universal PCR master mix (Applied Biosystems, Inc, catalog number 4364338), 1 microliter of 20× primer pair sold under the tradename TAQMAN gene expression assay (Applied Biosystems, Inc, catalog number 4331182), 1 microliter of template DNA and 8 microliter RNase-free water (Sigma-Aldrich, catalog number W4502). The specific gene expression assay kits used were FoxA2 (assay ID:Hs 00232764_ml), Sox17 (assay ID: Hs 00751752_ml), alpha fetoprotein (AFP, assay ID: Hs00173490_ml), transthyretin (TTR, assay ID:Hs00174914_ml), albumin (assay ID: Hs00910225_ml), hepatocyte nuclear factor (HNF) 4alpha (assay ID: Hs00230853_ml), tyrosine aminotransferase (TAT, assay ID: Hs00356930_ml), cytochrome P (CYP) 3a (Assay ID: Hs 00604506_ml) and GAPDH (assay ID: Hs99999905_ml) as normalization gene. Amplifications were performed starting with UNG activation step at 50° C. for 2 minutes followed by 10-minute template denaturation at 95° C. 40 cycles of denaturation at 95° C. for 15 seconds and combined primer annealing/extension at 60° C. for 1 minute were carried out.

Induced hepatocytes were also processed for immunostaining for hepatic markers. Briefly, differentiated cells cultured on 12-well plates were washed with PBS and fixed with 2.2% paraformaldehyde for 20 minutes at room temperature. Fixed cells were washed twice with PBS, followed by incubation at room temperature, for 1 hour with primary antibodies in blocking/permeabilization buffer (PBS with 0.3% Triton X-100 and 3% goat serum). Stained cells were washed three times in blocking/permeabilization buffer before incubation with the appropriate fluorophore-conjugated secondary antibodies. After the final wash (five times in washing buffer), the stained cells were examined by fluorescence inverted microscope.

### Results

Human kidney-derived iPS cells, clone RV4-5, were cultured in the presence of activin A and Wnt3a. After treatment with activin and Wnt3a for 3 days, RNA was extracted and various hepatocytes markers were determined by qRT-PCR.

Transcript levels for endoderm (FoxA2 and Sox17), primary hepatocyte (AFP and TTR), intermediate hepatocyte (albumin and HNF4 alpha), and mature hepatocyte (TAT and Cyp7a) markers from cells at different stages (day 0, 3, 9 and 17) are shown. The transcript level is expressed as fold-increase over the control cells (undifferentiated iPS cell at day 0). Values marked with an asterisk (*) indicate that this gene's average threshold cycle is high in undifferentiated control and is low in the test sample. This suggests that the actual fold-change value is at least as large as the calculated fold change result. Values marked with a hash mark (#) indicate that this gene's average threshold cycle is high but its relative expression level is low in both undifferentiated control and test samples.

As shown in Table 5, the induced cells showed a significant increase in Sox17 and Fox2a transcripts as determined by qRT-PCR. Interestingly HNF4 alpha transcript (but not protein) is also detectable at this stage. These results show that the human kidney-derived iPS cells were induced towards the definitive endoderm lineage.

TABLE 5

| | Differentiation Stage | | | |
|---|---|---|---|---|
| | Undiffer- | | Primary | Mature |
| | entiated | Endoderm | hepatocytes | hepatocytes |
| Transcripts | Day 0 | Day 3 | Day 9 | Day 17 |
| FOXA2 | 1 | 32.2 | 17.81 | 5 |
| SOX 17 | 1 | 312.18 | 52.26 | 7.02 |
| AFP | undetectable | undetectable | 5822.41* | 4275.27* |
| TTR | undetectable | undetectable | 12754.33* | 3563.06* |
| (Proalbumin) | | | | |
| Albumin | undetectable | undetectable | 11.33# | 5.56# |
| HNF4 alpha | undetectable | 34.36* | 606.94* | 31.1* |
| TAT | undetectable | undetectable | undetectable | undetectable |
| CYP7a | undetectable | undetectable | undetectable | undetectable |

Human kidney-derived iPS cells induced into definitive endoderm cells were further induced to become hepatocytes with two different media formulations for 7 days each. After treatment with the differentiation medium for 6 days, cells differentiated to early-intermediate hepatocytes. Cells at this stage expressed a high level of AFP, TTR and HNF 4 alpha transcripts. On the other hand, Sox17 and FoxA2 transcripts started to decrease (Table 5). Immunostaining shows that cells stained positive for alpha-feto protein (˜20%), TTR (approximately ˜40%) and HNF4 alpha (approximately 40%) at day 6. On day 17, the cells express higher level of alpha-feto protein and there is a moderate increase in TTR. Interestingly, there is a reduction in HNF 4 alpha transcripts (Table 3) and the number of cells stained positive with HNF 4 alpha.

### Example 7

### Hematoendothelial Differentiation of Human Kidney-Derived iPS Cells in OP9 Coculture

### Cell Culture

The human kidney-derived iPS cells (clone RV4-5, passage 28) was maintained in an undifferentiated state by weekly passage on human embryonic stem cell-qualified basement membrane matrix, sold under the tradename GELTREX (Invitrogen Corporation, catalog number A1048001) in feeder independent culture medium, sold under the tradename MTESR1 medium (STEMCELL Technologies, Inc., catalog number 05850). The OP9 mouse bone marrow stromal cell line was obtained from ATCC (American Tissue Culture Collection, Manassas, Va., catalog number CRL2749). This cell line was maintained on flasks coated with gelatin, sold under the tradename ESGRO, Millipore Corporation, catalog number SF008) in OP9 growth medium consisting of alpha-modified minimum essential media (alpha-MEM, Invitrogen Corporation, catalog number A1049001) supplemented with 20% non-heat-inactivated defined fetal bovine serum (FBS, Invitrogen Corporation, catalog number 16000-044).

Hematopoietic Differentiation of Human Kidney-Derived iPS Cells in Coculture with OP9 Cells

For cell differentiation, OP9 cells were plated onto flasks sold under the tradename CELLBIND SURFACE HYPERFLASK M Cell Culture Vessel (Corning Inc., Lowell, Mass., catalog number 10020) coated with ESGRO gelatin solution in OP9 growth medium. After formation of confluent cultures on day 4, half of the medium was changed, and cells were cultured for an additional 4 days. Human kidney-derived iPS cells were harvested by treatment with 1 milligram/milliliter collagenase IV (Invitrogen Corporation, catalog number 17104-019) and dispersed by scraping to maintain the cells in small clumps. Concurrently, human kidney-derived iPS cells cultures growing under the same conditions were used to obtain single cell suspension for counting. The human kidney-derived iPS cells were added to OP9 cultures at a density of 4.7×10⁴ cells/cm² in alpha-MEM supplemented with 10% FBS (HYCLONE FBS), 50 milligrams/milliliter ascorbic acid solution and 100 micromolar monothioglycerol (MTG; Sigma-Aldrich). The human kidney-derived iPS cells/OP9 cocultures were incubated for 10 days at 37° C. in normoxic conditions and 5% CO₂ with a half-medium change on days 4, 6, and 8. Cells were harvested at day 10, and single-cell suspension was prepared by treatment of the human kidney-derived iPS cells/OP9 cocultures with collagenase IV (Invitrogen Corporation; 1 milligram/milliliter in alpha-MEM) for 20 minutes at 37° C., followed by treatment with 0.05% trypsin-0.5 millimolar EDTA (ethylenediaminetetraacetic acid, Invitrogen Corporation) for 15 minutes at 37° C. Cells were washed twice with phosphate-buffered saline (PBS) containing 2% FBS, filtered through a 100-micron cell strainer (BD Biosciences, Palo Alto, Calif., catalog number 352360), counted, and used for flow-cytometric assays.

### Phenotype Analysis by Flow Cytometry

Cells were pre-stained with a cell viability stain, sold under the tradename LIVE/DEAD Fixable Near-IR Dead Cell Stain Kit (Invitrogen Corporation, catalog number L10119) to analyze only live cells. Cells were prepared in PBS containing 0.05% sodium azide, 1 mM EDTA, 2% FBS, Fc receptor blocking solution sold under the tradename HUMAN TRUSTAIN FCX (BioLegend, Inc., San Diego, Calif., catalog number 422301) and 2% normal mouse serum (Sigma-Aldrich, catalog number L2280) and were labeled with a combination of monoclonal antibodies (mAbs). Samples were analyzed using a FACS LSR11 flow cytometer (Becton Dickinson Immunocytometry Systems [BDIS], San Jose, Calif.) with FACSDIVA acquisition software (BDIS). List mode files were analyzed by FlowJo software (Tree Star, Ashland, Oreg.). The following mAbs were used: CD43-FITC, TRA-1-85-PE, CD117-PerCP/Cy5.5, CD34-PE/Cy7, CD31-APC, CD45-AmCyan, FLk-1-V450. Control staining with appropriate isotype-matched control mAbs (BD Pharmingen) were included to establish thresholds for positive staining

### Results

Human kidney-derived iPS cells maintained strictly in an undifferentiated state did not express CD34, CD31, CD43, or CD45 relative to antibody isotype controls. Both Flk-1 and CD117, which are known to be expressed on primitive hematopoietic progenitors, were found to be expressed on undifferentiated human kidney-derived iPS cells. In the OP9 coculture, approximately 11% of the viable human kidney-derived iPS cells (TRA-1-85 positive) were CD34⁺ cells. The human kidney-derived iPS cells differentiated into endothelial cells and hematopoietic progenitors can be identified by the expression of a common hematoendothelial marker, CD31 (PECAM-1). After 10 days of co-culture, 11.44% of the human kidney-derived iPS cells were CD31⁺ and 89% of the CD34⁺ cells were CD31 (hematoendothelial marker), which is commonly observed in hES differentiation into CD34+ cells (Vodyanik, M. A., and Sluvin, Il, Curr Protoc Cell Biol Chapter 23: Unit 23-26 (2007). Hematopoietic progenitors were distinguished from endothelial cells by CD43 (leukosialin; pan-hematopoietic marker) expression. After 10 days of co-culture, CD43 was present on 8% of the human kidney-derived iPS cells with 4% being CD31⁺CD43⁻ (endothelial potential) and 7% CD31⁺CD43⁺ (hematopoietic potential). In addition, 5% of the human kidney-derived iPS cells cocultured with OP9 cells were CD34⁺CD43⁺, which have multi-lineage hematopoietic potential and are capable of differentiation toward all blood lineages as well as B lymphoid cells. The commonly used CD45 pan-hematopoietic marker was not expressed on the CD34⁺ cells and CD117 and Flk-1 were also low in the CD34+ cells.

### Example 8

### Endodermal Differentiation of Human Kidney-Derived iPS Cells

### Endodermal Differentiation of Human Kidney-Derived iPS Cells

Single cells (human kidney-derived iPS cells prepared in Example 1, clone RV4-5, were plated onto GELTREX-coated 12 well plates at 105,000 vc/cm². After 3 days in MTESR1 media the cells were treated with a TGF-beta superfamily protein for three consecutive days in RPMI 1640 medium with 0.1% fatty acid-free bovine serum albumin (FAF-BSA, Proliant Health and Biologicals. Ankeny, Iowa, catalog number 68700) and CHIR99021 (glycogen synthase kinase 3 inhibitor, Stemgent, Inc. catalog number 04-0004).

### Phenotypic Analysis of Differentiated Human Kidney-Derived iPS Cells

Cells were removed from the 12 well plates by ACCUTASE and were analyzed for phenotypic markers presentative for endodermal differentiation. Cells were pre-stained with live/dead near-infrared (Invitrogen Corporation) allowing to analyze only live cells. Cells were prepared in PBS containing 0.05% sodium azide, 1 millimolar EDTA, 2% FBS, HUMAN TRUSTAIN FCX (Fc Receptor Blocking Solution) and 2% normal mouse serum (Sigma-Aldrich). Cells were surface stained with phycoerythrin (PE)-conjugated antibody to CXCR4 (BIOLEGEND, Inc., catalog number 306506). Cells were fixed, permeabilized and stained with allophycocyanin (APC)-conjugated antibody to SOX17 (R&D Systems Inc., catalog number IC1924A). CXCR4 (mesoendodermal marker) and SOX17 (definitive endodermal marker) were chosen as these markers have been used to elucidate definitive endodermal differentiation in pluripotent. cells (D'Amour, K. A. et al., Nat Biotechnol 23(12): 1534-1541 (2005); Spence, J. R. et al., Nature 470(7332): 105-109 (2011)). Control staining with appropriate isotype-matched control antibodies were included to establish thresholds for positive staining Samples were analyzed using a flow cytometer (Becton Dickinson Immunocytometry Systems, San Jose, Calif.) and acquired using the flow cytometry software sold under the tradename FACSDIVA acquisition software (Becton Dickinson Immunocytometry Systems). List mode files were analyzed by a flow cytometry analysis software sold under the tradename FLOWJO (Tree Star, Inc., Ashland, Oreg.).

### Results

Human kidney-derived iPS cells differentiated towards definitive endoderm lead to 80% of the viable cells being positive for SOX17 (definitive endodermal marker). SOX17 is not expressed in the other cell lineages (mesoderm, ectoderm, trophectoderm); thus the cells that express SOX17 protein are of definitive endoderm lineage. 36% of the cells were double positive for SOX17 and CXCR4 (mesoendodermal marker). Although CXCR4 has been reported in the mesoderm there were no CXCR4⁺SOX17⁻ cells, further demonstrating the cells are definitive endodermal cells. Undifferentiated iPS cell showed no evidence of definitive endoderm differentiation due to negative expression of SOX17 and CXCR4.

### Example 9

### Hepatocytes Differentiated from Human Kidney-Derived iPS Cells Transplantation into Fah^{-/-}Rag2^{-/-} Mice

Fah^{-/-} mice are defective in tyrosine metabolism and require 2-(2-nitro-4-trifluoro-methylbenzyol)-1,3-cyclohexanedione (NTBC) supply for survival. After NTBC withdrawal (NTBC-off), Fah^{-/-} mice undergo liver failure and death. They can be rescued by transplantation of wild-type primary hepatocytes, representing a useful model to characterize in vivo repopulation and functions of hepatocytes differentiated from human kidney-derived iPS cells. Immunodeficient Fah^{-/-}Rag2^{-/-} mice are used for transplantation to reduce the likelihood of immunological rejection (Huang, P. et al., Nature 475: 386-389 (2011)).

Fah^{-/-}Rag2^{-/-} mice are maintained with 7.5 milligrams/liter NTBC in the drinking water. Hepatocytes differentiated from human kidney-derived iPS cells are transplanted into the spleens of Fah^{-/-}Rag2^{-/-} mice at the age of 8-12 weeks. NTBC is withdrawn from the drinking water after cell transplantation. Fah^{-/-}Rag2^{-/-} mice without any transplantation also have NTBC withdrawn as a control. A survival curve is generated by SPSS for windows using Kaplan-Meier method. Eight weeks after transplantation, the blood of surviving cell-transplanted Fah^{-/-}Rag2^{-/-} mice is collected from the retro-orbital sinus and centrifuged at 12,000 rpm for 15 minutes. The serum is frozen at −80° C. until biochemical analyses. Total bilirubin, albumin, blood urea nitrogen and creatinine are measured. After blood collection, mice are killed by cervical dislocation and livers are harvested, fixed and stained with haematoxylin and eosin. Blood and liver samples of control NTBC-off Fah^{-/-}Rag2^{-/-} mice are collected after losing 20% body weight.

### Example 10

### mRNA-Mediated Reprogramming of hKDC into iPS Cells

hKDC, obtained according to the methods described in US Patent Publication Number 2008/0112939, were transduced with mRNA constructs from Stemgent, Inc. (San Diego, Calif., catalog number 00-0067), specifically mRNA encoding for the human transcription factors OCT4, SOX2, KLF4, c-MYC, and LIN28.

hKDC were thawed and cultured for one passage before transduction. One day before transduction, hKDC were trypsinized and plated onto a 6-well plate (pre-seeded with inactivated human newborn foreskin fibroblasts (Globalstem Incorporated, Rockville, Md. catalog number GSC-3001G or GSC-3001M) at 2.5×10⁴ cells per well in 2 milliliters of renal epithelial growth medium (REGM, Lonza Walkersville, Inc., Walkersville, Md.) per well. Cells were incubated overnight at 5% CO₂ and 37° C. Human newborn foreskin fibroblast (NuFF) feeder plates were prepared 24 hours prior to use by seeding NuFF at a density of 2.5×10⁵ in NuFF culture medium on 6-well plates pre-coated with 0.1% gelatin.

On day 1, REGM was aspirated and replaced with 2 milliliters of optimized reprogramming medium sold under the tradename PLURITON mRNA Reprogramming medium (Stemgent, Inc., catalog number 00-0070 supplemented with 1× of penicillin/streptomycin (Invitrogen Corporation, catalog number 15070-063) containing 200 nanograms/milliliter of B18R (type I interferon receptor, eBioscience, Inc., San Diego, Calif., catalog number 34-8185-85) and incubated at 5% CO₂ and 37° C. for 4 hours. The mRNA transfection complex was prepared by adding 200 microliters of a reduced serum culture medium sold under the tradename OPTI-MEM (Invitrogen Corporation, Catalog number 31985-070) to a vial containing 50 microliters of mRNA cocktail and mixed gently. A separate tube was prepared by gently mixing 225 microliters of OPTI-MEM and 25 microliters of a transfection reagent sold under the tradename LIPOFECTAMINE RNAIMAX (Invitrogen Corporation, catalog number 13778075). The contents of the two tubes were combined and incubated at room temperature for 15 minutes to allow the mRNA to complex with the transfection reagent. To transfect the hKDC, 120 microliters of the mRNA transfection was added in a dropwise fashion to each well. The plate was gently rocked to distribute the mRNA transfection complex and then the plate was incubated at 5% CO₂ and 37° C. for 4 hours. Afterwards, the culture medium containing the mRNA transfection complex was aspirated and replaced with 2 milliliters of PLURITON reprogramming medium containing 200 nanograms/milliliter B18R and incubated overnight at 5% CO₂ and 37° C.

The transfection step was repeated 4 more times on days 2-5. On days 6-17, the transfection was repeated for 12 more times and on these days, the cells were maintained in NuFF-conditioned medium. NuFF-conditioned medium was generated by plating inactivated NuFF on a T75 tissue culture flask (pre-coated with 0.1% gelatin solution) at a density of 4×10⁶ cells in 25 milliliters of medium containing DMEM (Invitrogen Corporation, catalog number 11965-092), 10% defined FBS (Atlas Biologicals, Inc., Fort Collins, Colo., catalog number F-0500-A), GLUTAMAX, and penicillin-streptomycin and incubated overnight at 5% CO₂ and 37° C. The culture medium was aspirated, cells washed once with 10 milliliter of PBS, and medium was replaced with 25 milliliters of PLURITON reprogramming medium (Stemgent Inc., catalog number 01-0015) supplemented with 4 nanograms/milliliter bFGF sold under the tradename STEMFACTOR (Stemgent, Inc., catalog number 03-0002) and 1× penicillin/streptomycin. After overnight incubation at 5% CO₂ and 37° C., the NuFF-conditioned medium was collected and stored at −20° C. Fresh PLURITON medium supplemented with 4 nanograms/milliliter STEMFACTOR basic FGF (Stemgent, Inc., catalog number 03-0002) and 1× penicillin/streptomycin was added, incubated overnight, and collected for five additional days yielding 150 milliliters of NuFF-conditioned medium. The collected aliquots were pooled, filter-sterilized using a 0.22 micron filter, and stored at −20° C. until use. Prior to use, PLURITON Supplement (2500×, Stemgent Inc., catalog number 01-0016) was added to 1× concentration.

During the transfection period, confluent cells were passaged to allow for further proliferation and iPS cell colony formation. To do this, cells were washed with PBS and harvested by adding 0.5 milliliter of Trypsin/EDTA for primary cells (ATCC, catalog number PCS-999-003) per well, and incubated for 5 minutes 5% CO₂ and 37° C. The side of the well was gently tapped to assist the dissociation and release of the cells and 0.5 milliliter of trypsin neutralizer (ATCC, catalog number PCS-999-004) was added to each well. The cells were collected by transferring to a 15 milliliter conical tube, washing the well with 1 milliliter of PLURITON reprogramming medium, and centrifuging at 200×g for 5 minutes. The cell pellet was resuspended in 1 milliliter of PLURITON reprogramming medium and seeded onto fresh NuFF feeder plate containing 2 milliliters of PLURITON reprogramming medium supplemented with 200 nanograms/milliliter B18R and 10 micromolar Y27632 (ROCK inhibitor, Stemgent Inc., catalog number 04-0012).

To monitor the formation of reprogrammed or iPS cell colonies, the transfected hKDC were incubated in NuFF-conditioned medium without B18R for 3 days to allow the colonies to expand. The primary iPS cell colonies were identified based on morphology and by sterile, live-staining with antibody sold under the tradename STAINALIVE DYLIGHT 488 Mouse anti-Human TRA1-81 (Stemgent, Inc., catalog number 09-0068). Colonies exhibiting the ‘classic’ reprogrammed or iPS cell morphology were manually picked and seeded onto a single well of a 12-well NuFF feeder plate. Culture medium was changed daily. After 4-6 days, the colonies were manually picked from the 12-well plates and expanded into 6-well plates. Culture medium was changed daily and manually split 1:3 every 4-6 days. Cells from each well were frozen in CRYOSTEM freezing medium.

### Results

Reprogramming of hKDC with the mRNA encoding the five reprogramming factors resulted in reprogrammed colonies exhibiting the iPS cell morphology and positive staining for TRA1-81.

### SUMMARY

Overall, we have shown the generation of human kidney-derived iPS cells by overexpression of human transcription factors using integrating (viral) and non-integrating (non-viral) methods. These results demonstrate that human kidney-derived iPS cells express the pluripotency markers TRA1-60, TRA1-81, SSEA3, SSEA4, and NANOG and exhibit positive alkaline phosphatase staining Upon examination of a 100-500 base pair region of the Oct4 promoter, the human kidney-derived iPS cells show a change in methylation on 7 methylation sites compared with the parental hKDC line.

These cells also display protein markers of cells derived from ectodermal, mesodermal, and endodermal lineages showing the differentiation potential of these reprogrammed cells. The expression of specific cell-specific markers suggest that after employing differentiation protocols, these cells can be differentiated into hepatocyte-like, hematoendothelial lineage, and definitive endoderm cells.

While the invention has been described and illustrated by reference to particular embodiments and examples, those of ordinary skill in the art will appreciate that the invention lends itself to variations not necessarily illustrated herein. For this reason, then, reference should be made solely to the appended claims for purposes of determining the true scope of the invention.

1.-5. (canceled)

6. A method of making a human kidney-derived induced pluripotent cell comprising the steps of:
providing a human kidney-derived cell, wherein the human kidney-derived cell is positive for the expression of HLA-I and CD 44 and at least one of Oct-4, Rex-1, Pax-2, Cadherin-11, FoxD1, WT1, Eya1, HNF3B, CXC-R4, Sox-17, EpoR, BMP2, BMP7, or GDF5; and negative for the expression of CD133 and E-cadherin and at least one of Sox2, FGF4, hTert, Wnt-4, SIX2 or GATA-4, transfecting the human kidney derived-cell with each one of a VSVg murine retrovirus expressing human transcription factor OCT4, a VSVg murine retrovirus expressing human transcription factor SOX2, a VSVg murine retrovirus expressing human transcription factor KLF4, and a VSVg murine retrovirus expressing human transcription factor c-MYC,
culturing the transfected human kidney-derived cell,
identifying an induced pluripotent stem cell,
isolating the induced pluripotent stem cell,
subculturing the induced pluripotent stem cell, and
providing an induced pluripotent stem cell.

7. The method of claim 6, further comprising transfecting the human kidney-derived cell with a VSVg murine retrovirus expressing human transcription factor p53-shRNA.

8. A method of making a human kidney-derived induced pluripotent cell comprising the steps of:
providing a human kidney-derived cell, transfecting the human kidney-derived cells with each one of an mRNA encoding a Oct-4 protein, an mRNA encoding a Sox2 protein, an mRNA encoding a Klf4 protein, an mRNA encoding a c-myc protein, and an mRNA encoding a Lin28 protein,
culturing the transfected human kidney-derived iPS cell,
identifying a induced pluripotent stem cell,
isolating the induced pluripotent stem cell,
subculturing the induced pluripotent stem cell, and
providing an induced pluripotent stem cell.

9. An induced pluripotent stem cell made by the method of claim 6.

10. An induced pluripotent stem cell made by the method of claim 8.
